(19) United States
(12) Patent Application Publication
(10) Pub. No.: US 2025/0128257 A1
(43) Pub. Date: Apr. 24, 2025

(54) FLUIDIC APPARATUS AND METHOD FOR SAMPLE PREPARATION BY MEANS OF A FLUIDIC APPARATUS

(71) Applicant: Technische Universität Dresden, Dresden (DE)

(72) Inventors: Andreas RICHTER, Dresden (DE); Denise GRUNER, Dresden (DE); Mohammed SHAHADHA, Dresden (DE); Anthony BECK, Dresden (DE); Franziska OBST, Dresden (DE); Markus FRIEDEMANN, Dresden (DE); Mario MENSCHIKOWSKI, Radeberg (DE)

(21) Appl. No.: 18/924,735

(22) Filed: Oct. 23, 2024

(30) Foreign Application Priority Data
Oct. 24, 2023 (DE) 10 2023 129 147.5

(51) Int. Cl. B01L 3/00; C12Q 1/6844; C12Q 1/70

(52) U.S. Cl. CPC B01L 3/502753; C12Q 1/6844; C12Q 1/701; B01L 2200/10; B01L 2200/16; B01L 2300/0681; B01L 2300/087; B01L 2300/088; B01L 2300/0883; B01L 2300/0887

(57) ABSTRACT

The disclosure relates to a fluidic apparatus having a first channel section and n second channel sections, wherein n∈N and n≥1, at least one opener element and at least one closer element. The apparatus according to the disclosure is characterized in that each of the n second channel sections is arranged so as to adjoin the first channel section by means of an opener element; each opener element is configured to establish a fluidic connection from the respective second channel section to the first channel section; the first and the n second channel sections each have a receiving region; a closer element is arranged in the first channel section upstream of at least one of the opener elements in the flow direction of a fluid.

### CROSS REFERENCE TO RELATED APPLICATION

The present application claims priority to German Patent Application No. 10 2023 129 147.5 filed on Oct. 24, 2023. The entire contents of the above-listed application are hereby incorporated by reference for all purposes.

### TECHNICAL FIELD

The disclosure relates to a fluidic apparatus having a first channel section and n second channel sections, wherein n∈□ and n≥1, at least one opener element and at least one closer element. The apparatus according to the disclosure is characterized in that.
each of the n second channel sections is arranged so as to adjoin the first channel section by means of an opener element;
each opener element is configured to establish a fluidic connection from the respective second channel section to the first channel section;
the first and the n second channel sections each have a receiving region,
a closer element is arranged in the first channel section upstream of at least one of the opener elements in the flow direction of a fluid.

### BACKGROUND

Sample preparation is very often a time-critical step in the rapid and accurate diagnosis of, for example, infectious diseases and is therefore essential for the initiation of successful therapy. For many diagnostic assays, sample preparation is currently performed manually (that is to say by a sequence of various manually performed pipetting steps) or, in combination with the assay to be carried out, in what are known as test cartridges or by means of a pipetting robot. Manual preparation of samples is time-consuming and prone to error and can generally be carried out only by specially trained personnel. Test cartridges and pipetting robots allow automated execution of the process steps but have only limited scalability in respect of throughput and require complex control by external devices.

### SUMMARY

Lab-on-a-chip (LOC) technology offers the possibility of drastically simplifying and automating sample preparation by integrating the various process steps on a microfluidic platform. They therefore represent an alternative to pipetting robots, which process larger sample quantities in comparison with microfluidics. Microfluidics offers the possibility of enabling even the smallest sample quantities to be processed and analysed. By virtue of the fact that assays are carried out in a compact, miniaturized format and the various process steps are integrated and parallelized on one platform, process times and costs (less process media, no specially trained personnel, no expensive equipment) can be reduced and process safety can be increased. This technology supports portability and point-of-care use. Since work is carried out in a closed system, the risks of cross contamination and hazards for the user are reduced.

However, since a sequence of different process steps has to be implemented for sample preparation, the complexity of current systems is relatively high. Thus, in the simplest case, the process media are applied sequentially, or, inter alia, pneumatic elements are used for sequential fluid transport (e.g. FilmArray from BioFire). This requires additional devices for controlling these elements.

Microfluidic systems for sample preparation are known in the prior art. Commercial products generally comprise a single-use article/disposable article (articles which are used once only), which is preloaded with the necessary reagents, and a transportable device which carries out the various process steps in an automated manner.

The FilmArray from BioFire comprises an injection-moulded polypropylene reservoir, which contains the biochemicals, and a region consisting of polyester/polypropylene, in which the microfluidic structures, such as channels and “blisters” are structured. Here, the “blisters” contain additional reagents. Ceramic particles are integrated for the purpose of cell lysis and magnetic silica particles are integrated for nucleic acid extraction. Lyophilized oligonucleotides are integrated for evaluation by means of polymerase chain reactions (PCR). The associated device contains two Peltier elements in order to implement the temperature programme of the PCR, and three pneumatic elements in order to control the transport of the fluids. Pistons push a plunger into the polypropylene reservoirs and thereby inject the reagents into the microfluidic structures. Silicone bladders, which are moved by means of the “blisters”, transport the liquids. During this process, the pistons, silicone bladders and seals are activated by electronically controlled valves in order to regulate the liquid flow in accordance with the process protocols.

The ID NOW test platform from Abbott comprises a sample receiver, a test base, a transfer cartridge and the ID NOW device. The sample receiver already contains elution/lysis buffer. The test base has two sealed reaction cylinders containing the appropriate lyophilized pellets for amplification (for the sample and internal control), and the transfer cartridge is responsible for transferring the eluted sample to the test base. The device performs heating and mixing steps as well as detection. The assay is carried out in three successive steps.

U.S. Pat. No. 10,814,321 discloses a method and a molecular-diagnostic apparatus for detecting, analysing and identifying genomic DNA. The cartridge used to transfer samples of genomic material comprises a separation and direction system. The transfer of the various reagents and of the sample is accomplished, inter alia, by means of capillary forces, pushing “push valves/thrust valves”, the application of a vacuum, or electrokinetic forces. In the said disclosure, the isolation of the genomic material is accomplished with the aid of a substrate which can bind and release the genomic material (“charge switch technology”).

US20200215544 discloses a self-contained apparatus for isolating nucleic acids, cell lysates and cell suspensions, which is used with an instrument. The apparatus comprises a macroscopic component, which contains a chamber for the unprocessed sample and storage reservoirs for the reagents. The microfluidic component, which is in communication with the macroscopic component, contains the matrix for nucleic acid purification. In addition, the apparatus has at least one interface with the drive mechanism on the instrument in order to drive the reagents through the microfluidic element and the matrix for nucleic acid purification. Here, the drive is pneumatic. Active or passive valves can be used for fluid control, with pneumatic control being preferred.

U.S. Pat. No. 10,947,528 discloses a microfluidic apparatus for extracting, isolating and analysing DNA from cells. This comprises an inlet opening for receiving the sample, an outlet opening for obtaining the isolated DNA, and a microfluidic channel, which comprises a micropillar array for trapping cells and for binding DNA. The cells and reagents are introduced into the microfluidic system by means of a pressure-controlled flow, with the syringe being replenished with the reagents accordingly.

U.S. Pat. No. 10,843,188 discloses an integrated system for processing microfluidic samples and a method for using the system. It comprises an apparatus and a microfluidic cartridge, which contains the reagents and a microfluidic network for processing the sample, wherein the microfluidic network also contains an arrangement for retaining the polynucleotides (nucleic acids). This retention arrangement comprises a polyalkylene imine or a polycationic polyamide in the form of one or more particles, which can also be taken from the microfluidic cartridge. Fluid control is accomplished by means of integrated phase change materials, e.g. eutectic alloys, wax, polymers, plastics and combinations thereof. For transporting liquids, use is made of actuators that generate a gas pressure.

U.S. Pat. No. 11,142,757 discloses a microfluidic cartridge for processing and detecting nucleic acids. In this case, extraction of the nucleic acids is accomplished by means of magnetic particles. The flow through a microfluidic channel is influenced and fluid control achieved by means of a series of external pins or other indenters, which are guided through valve guides.

U.S. Pat. No. 10,940,473 discloses a microfluidic apparatus for nucleic acid analysis. This comprises at least one first microfluidic chamber and at least one first microfluidic channel for transporting the sample. A second microfluidic channel transports the lysis chemicals to the first microfluidic chamber, wherein the isolation and purification of the nucleic acids is accomplished by means of affinity beads. Fluid control is accomplished by means of elastic valves, wherein the application of a pressure (gas or liquid) to a fluidic channel leads to deflection of an elastic membrane, which blocks or hinders the fluid flow in a channel underneath it.

EP2836302 discloses a method and an apparatus for targeted process control in a microfluidic system comprising integrated active elements for carrying out volumetric defined mixing reactions in defined time sequences. In this case, a chamber which is divided by a soluble membrane is filled in parallel with two or more liquids. The integrated active elements ensure the volume definition and time definition of the mixing reaction. The disclosed method and the apparatus thus allow targeted process control by means of integrated active elements that allow or prevent a fluid flow by interaction with the process media. In this case, however, only the method for mixing a first and a second liquid is disclosed. The transport channels of the device are arranged in parallel for this purpose and end in a mixing chamber. However, the device does not enable the sequential provision of fluids in a microfluidic device. Nor is a method for the sequence of a process control operation for sample preparation provided. In addition, this patent does not describe any components that allow binding of at least one component of the sample (immobilization of components of a sample).

DE112007003160 discloses an automatic microfluidic processor with integrated active elements, which, by logical combination of the active individual elements, combines component tasks of a defined procedure into a sequence of tasks and defines the activation times and additional parameters of the individual elements by means of the processor design. The intention is thereby to implement more or less complex biological, biochemical or chemical processes on an automatic microfluidic processor. Admittedly, the apparatus disclosed enables component procedures that are part of, for example, chemical, biochemical or biological processes to be executed by logical combination of the active component elements. However, this allows only processes for detecting, for example, biological components but not a method for sample preparation. In addition, it does not describe the integration of a trap region, e.g. for binding nucleic acids.

It is therefore the object of the present disclosure to provide an apparatus and a method which serve for carrying out a complex multistage protocol for sample preparation in an automated manner without external control.

For this purpose, the disclosure provides an apparatus according to Claim 1 and a method according to Claim 12.

### DESCRIPTION

The fluidic apparatus according to the disclosure has a first channel section and n second channel sections, wherein n∈□ and n≥1, at least one opener element and at least one closer element.

According to the disclosure, each of the n second channel sections is arranged so as to adjoin the first channel section by means of an opener element. In one embodiment, each of the n second channel sections is arranged peripherally with respect to the first channel section. In particular, in one embodiment, each of the second channel sections is arranged approximately parallel to the first channel section in the region of the opener element. According to the disclosure, each of the opener elements is configured to establish a fluidic connection from the respective second channel section to the first channel section. In addition, the first and the n second channel sections each have a receiving region. That is to say that the first channel section has a receiving region and each of the n second channel sections likewise has a respective receiving region.

According to the disclosure, a closer element is arranged in the first channel section upstream of at least one of the opener elements in the flow direction of a fluid. In one embodiment of the disclosure, a respective closer element is arranged in the first channel section upstream of each opener element in the flow direction of a fluid.

The flow in the first channel section forms from the receiving region in the direction of the closer element in the first channel section and continues along the first channel section. According to the disclosure, the flow direction of a fluid in the first channel section is therefore understood to mean the flow of the fluid from the receiving region towards the closer element and onwards along the first channel section. In each second and each further channel section, the flow of a fluid forms from the receiving region of the second or the further channel section in the direction of the opener element in the second or further channel section and continues along the second or further channel section. According to the disclosure, the flow direction of a fluid in each second and each further channel section is therefore understood to mean the flow of the fluid from the receiving region of the second or the further channel section towards the opener element and onwards along the second or the further channel section.

In one embodiment, the receiving region of the first channel section is followed in the first channel section by a closer element and then an opener element, by means of which a second channel section adjoins the first channel section. Further second channel sections can subsequently be arranged adjoining the first channel section by way of further opener elements. In one embodiment, a respective further closer element is arranged between each of the further opener elements in the first channel section.

The term “channel section” includes both rectangular channels and channels with a circular cross section (hoses). The channel diameters are usually in the range of from 50 μm to 1 mm, preferably in the range of from 100 μm to 500 μm, particularly preferably in the range of from 150 μm to 300 μm. Channels of rectangular cross section accordingly have dimensions which are comparable with the channel diameters described. Channel sections within the meaning of the disclosure are not mixing or reaction chambers in which a mixing reaction between different fluids takes place. In particular, no significant and thus functionally relevant mixing of fluids from the first and/or second channel sections takes place in the first channel section. The channel sections are used for the sequential transport of fluids in the device according to the disclosure.

In one embodiment, the fluidic apparatus has n≥2 second channel sections. According to the disclosure, each of the n second channel sections is arranged spaced apart from one another in series on the first channel section. This is to be understood as meaning that the n second channel sections are arranged in the form of a series connection on the first channel section. Spaced apart or also spatially spaced apart describes according to the disclosure that the second channel sections are arranged one behind the other in the direction of flow of a fluid in the first channel section on the first channel section. If, for example, a first second channel section is adjacent to the first channel section, the next adjacent second channel section is arranged at a spatial distance from the first second channel section on the first channel section in the direction of flow of a fluid in the first channel section. This applies analogously for each further second channel section. Particularly preferably, there are no overlapping areas between different second channel sections. This means that a fluid from a first second channel section and a fluid from a further second channel section are passed through the first channel section separately in time, i.e. sequentially. Although slight mixing due to diffusion or turbulence may occur at the interfaces of successive fluids, this should be as low as possible and in no way represents a complete mixing process of two fluids, as occurs in a mixing chamber. In particular, no significant and thus functionally relevant mixing of fluids from the first and/or second channel sections takes place in the first channel section according to the disclosure.

The fluidic apparatus according to the disclosure is preferably set up to transport fluids sequentially through the first channel section.

In one embodiment, the fluidic apparatus furthermore has m third channel sections, wherein m∈□ and m≥1, wherein each of the m third channel sections is arranged so as to adjoin at least one of the n second channel sections by means of a respective opener element. According to the disclosure, the respective opener element is configured to establish a fluidic connection from a third channel section to a second channel section. As a particular preference, each of the m third channel sections has a dedicated receiving region.

In another embodiment, the fluidic apparatus furthermore has ƒ k^{th} channel sections, wherein ƒ∈□ and ƒ≥1 and k∈□ and k≥4. Each of the ƒ k^{th} channel sections is arranged so as to adjoin at least one of the (k−1)^{th} channel sections by means of a respective opener element, wherein the respective opener element is configured to establish a fluidic connection from a k^{th} channel section to a (k−1)^{th} channel section. In this embodiment, the fluidic apparatus can therefore have ƒ fourth channel sections, for example, wherein each of the ƒ fourth channel sections adjoins a third channel section by means of a respective opener element. For the purposes of the disclosure, it is furthermore possible in addition for there to be ƒ fifth channel sections, wherein each of the ƒ fifth channel sections adjoins a fourth channel section by means of a respective opener element etc. As a particular preference, each of the ƒ k^{th} channel sections has a dedicated receiving region.

In one embodiment, k is preferably 4, 5, 6, 7, 8, 9 or 10.

All the characteristics that have been described for the n second channel sections apply equally to the m third and ƒ k^{th} channel sections.

The closer elements and opener elements are designed in such a way that they can each adopt an open state and a closed state. In the present disclosure, the opener and closer elements perform the function of valves. In the open state, a fluid can flow through the opened opener element or closer element. The opened opener element or closer element acts like an opened valve. In the closed state of the opener element or closer element, it is not possible for a fluid to flow through the opener element or closer element. The closed opener element or closer element thus forms a barrier and acts like a closed valve.

### Closer Element

In a preferred embodiment of the present disclosure, the closer elements are embodied as swelling material barriers that can be activated by fluid contact. When the closer elements are wetted by a fluid in the flow path, there is an increase in the volume of the closer element due to fluid absorption, as a result of which the flow path in the channel section in which the closer element is arranged is increasingly constricted until, on account of the complete filling of the cross section of the channel section, there is a discontinuation of the flow in the flow path and consequently an interruption of the flow.

The closer element embodied as a swelling material barrier is introduced in the dry state into a channel section of the fluidic apparatus. After the increase in the volume of the closer element due to fluid absorption has taken place, the closer element and thus the swelling material barrier remains in the swollen state. This means that no reversal of the swelling takes place after the increase in volume, as a result of which the swelling material barrier undergoes activation by fluid absorption only once. This is advantageous, in particular, since, for example, the volume of a fluid in the microfluidic apparatus can be defined by means of the closer element.

In one embodiment of the disclosure, the closer elements comprise hydrogels or consist of the latter. The hydrogels are preferably chemically and/or physically cross-linkable. For the purposes of the disclosure, hydrogels are understood to mean a polymer that contains water but is insoluble in water, the molecules of which are connected by chemical, e.g. covalent, bonds, or physically, e.g. by intermeshing of the polymer chains, to form a three-dimensional network. By virtue of incorporated hydrophilic polymer components, they swell in liquids with a considerable increase in volume, but without losing their physical cohesion. According to the disclosure, the hydrogels are designed in such a way that they remain in the swollen state after contact with fluids.

In another embodiment of the disclosure, the closer elements comprise hydrogels that are selected from a group comprising polyacrylamides, polyvinyl alcohols, polyacrylates, hydroxy cellulose compounds, polyvinylpyridines or polyglycols (e.g. polyethylene glycol, polypropylene glycol) and derivatives thereof or consist thereof.

For the purposes of the disclosure, “activation” of the closer element is understood to mean that the closer element makes a transition from the open to the closed state. Activation begins with contact between the closer element and a fluid, which initiates the swelling of the closer element. The activation time describes the time period from activation until the closer element has swollen to such an extent that the flow path is completely closed by the closer element. If the closer element is embodied as a swelling material barrier, this can be activated by contact with a fluid, which leads to swelling of the closer element. Advantageously, the time which the closer element requires to make the transition from the open to the closed state can be precisely defined by
the materials used and the composition thereof; and/or
the geometry of the closer element; and/or
the type of fluid which activates the closer element; and/or
the pressure or volume flow in the channel section; and/or
the temperature; and/or
the geometry of the surrounding channel section or of the surrounding structure support which forms the channel section.

The effect of the geometry of the surrounding channel section or of the surrounding structure support which forms the channel section is described by the seat size. The closer element is surrounded by walls of a channel and/or structure support, which make available a free space of defined size into which the closer element is introduced. The free space into which the closer element is introduced is referred to as the seat size. The dimensions of the closer element in the swollen state are preferably larger than the dimensions of the seat size, and therefore the closer element is pressed against the channel walls or walls of the structure support in the swollen state and thus closes the channel section.

If the closer element comprises hydrogels, it is possible, in a preferred embodiment, for the closer elements to be produced from the hydrogels in swollen form. For this purpose, use is made of moulds, the diameter and height of which are greater than the subsequently used seat size (valve seat diameter and channel height). After these closer elements have dried, their diameter and height is smaller than the seat size, and the closer elements can be introduced into a channel section without problems. The geometry of the closer element can be adjusted accordingly during production.

In one embodiment, the time which a closer element requires to transfer from the open to the closed state is adjusted, in particular, by varying the seat size.

### Opener Elements

In one preferred embodiment, opener elements are designed as soluble barriers that serve as fluid-soluble valves. Dissolution of the barrier is achieved by wetting the opener element with a fluid in the flow path. The dissolution of the barrier results in an increase in the through flow, and a first flow path in one channel section and a second flow path in a further channel section are connected fluidically to one another.

In another embodiment of the disclosure, the opener elements are made of un-cross-linked polymers, salts or organic natural substances such as saccharides. This is the case when the active elements are embodied as liquid-soluble barriers. In this context, it is possible to use all materials which form a solid, sol-gel or the like in the dried state and go into solution upon contact with a liquid. In principle, the material basis of the un-cross-linked polymers can be the same as for the cross-linked polymers of the closer elements. Whereas polymers cross-linked to form a three-dimensional network serve as swellable swelling material barriers, the same polymers dissolve in the liquid if they are un-cross-linked since the polymer chains which are not interlinked go into solution.

In one embodiment of the disclosure, the opener elements comprise soluble polymers selected from a group comprising polyvinyl alcohols, hydroxy celluloses or polyglycols (e.g. polyethylene glycol, polypropylene glycol) and derivatives thereof or consisting of these.

In another embodiment of the disclosure, the opener elements comprise polyglycols, mixtures of polyglycols of various chain lengths or mixtures of polyglycols of various chain length and additives, e.g. mannose or cellulose fibres.

In one embodiment, the opener element has at least one pair of connecting elements and a channel-shaped part. The pair of connecting elements comprises a first and a second connecting element, which produce a fluid connection between a first channel section and a second channel section when the opener element is in the open state. In this case, a fluidic connection is produced between a first channel section and the opener element by the first connecting element, and a fluidic connection is produced between a second channel section and the opener element by the second connecting element. The connecting elements of this embodiment of the opener elements therefore always occur as a pair of connecting elements.

Here, each connecting element of the pair of connecting elements forms at least one sudden expansion of the opener element in the direction of a channel section, wherein, according to the disclosure, a sudden expansion can be a channel-shaped connection to a channel section.

According to the disclosure, each sudden expansion of a connecting element adjoins a channel section. According to the disclosure, the outer wall of the sudden expansion forms what is referred to as an opening angle a with respect to the outer wall of the adjoining channel section. According to the disclosure, the opening angle is α≤90°.

In one embodiment with connecting elements, at least one sudden expansion of a connecting element is furthermore oriented orthogonally with respect to the channel-shaped section of the opener element.

In one embodiment of the present disclosure, the channel-shaped part of the opener element has a chamber-shaped part. The chamber-shaped part can, for example, have the shape of a cuboid, a cylinder or an elliptical cylinder. Here, the height of the chamber-shaped part corresponds to the height of the channel-shaped part of the opener element. In this embodiment, the at least one sudden expansion and each further sudden expansion of a connecting element adjoins the chamber-shaped expansion of the channel-shaped part of the opener element.

In one embodiment of the present disclosure, a connecting element has a plurality of sudden expansions. A connecting element has, for example, 1 to 30, preferably 1 to 20, particularly preferably 1 to 10, sudden expansions. Each sudden expansion preferably has the same geometry.

As a particular preference, the at least two connecting elements which form a pair of connecting elements and thus establish a fluidic connection between a first and a second channel section have the same number of sudden expansions.

In one embodiment of the present disclosure, the first and second connecting elements of a pair of connecting elements are arranged opposite one another. In addition, as a particular preference, all the sudden expansions of the first connecting element are arranged opposite a sudden expansion of the second connecting element in each case, that is to say in pairs. In another embodiment, the first and second connecting elements of a pair of connecting elements are arranged parallel to one another.

For the purposes of the disclosure, “activation of the opener element” is understood to mean that the opener element makes a transition from the closed to the open state. If the opener element is embodied as a soluble barrier, this can be activated by contact with a fluid, leading to the dissolution of the opener element. Activation begins with the contact of the opener element with a fluid, initiating the opening of the opener element, e.g. by dissolving a soluble barrier. The activation time describes the time period from activation until the opener element is open, with the flow path through the opener element thus being open. Advantageously, the time which the opener element requires to make the transition from the closed to the open state can be precisely defined by
the materials used and the composition thereof; and/or
the geometry of the opener element; and/or
the type of fluid which activates the opener element; and/or
the pressure or volume flow in the channel section; and/or
the temperature; and/or
the geometry of the surrounding channel section or of the surrounding structure support which forms the channel section, e.g. in the form of the step width.

The opener element is surrounded by walls of a channel and/or structure support, which make available a free space of defined size (e.g. hole, cavity), into which the opener element is introduced. In one embodiment, the time which the opener element requires to make the transition from the closed to the open state can be influenced by means of the diameter of the hole and the height of the opener element (e.g. determined by the height of the structure support or the thickness of the membrane if the opener element is configured as a membrane) or the step width. The thicker the opener element or the wider the step, the more material the opener element has and the more time is required for the dissolution of the barrier.

Both the opener elements and the closer elements according to the present disclosure are thus activated without auxiliary energy. For the purposes of the present disclosure, “without auxiliary energy” is understood to mean the omission of the supply of energy from an externally controlled electric or thermal energy source to the opener and closer elements according to the disclosure. By virtue of the performance of functions by the opener and closer elements according to the disclosure in a manner that allows definition in terms of time and without auxiliary energy, these elements are advantageously suitable for use as valves in autonomous microfluidic apparatuses.

As already described, it is possible, by varying the construction of the opener and closer elements, to exert a direct influence on the time sequence of the fluidic contacting and the dosing of the fluids in a microfluidic apparatus. It is possible, for example, by a suitable choice of materials and of the dimensioning of the opener and closer elements, to influence the time response in the fluidic apparatus.

The number of channel sections and thus of opener and closer elements in the fluidic apparatus depends on the complexity of the application, e.g. on the number of assays used. In particular, the number of fluidic method steps required plays a decisive role here.

### Trap Element

In one embodiment of the present disclosure, the first channel section furthermore has a trap element, which is arranged in the first channel section downstream of the last opener element in the flow direction of a fluid, which connects one of the n second channel sections to the first channel section.

For the purposes of the disclosure, a “trap element” is understood to mean a region which connects the flow path in the first channel section upstream of the trap element to the flow path in the first channel section downstream of the trap element via a permeable element, wherein the two flow paths have a common wall in the trap element.

In a preferred embodiment, the trap element is a permeable element. In one embodiment, this can be designed as a porous element with a plurality of openings. Suitable porous elements are selected from the group comprising filters, porous membranes or a gel matrix. These provide numerous openings in the form of pores and/or channels. In a preferred embodiment, cellulose membranes, silica membranes or integrated hydrogel particles are used.

As integrated hydrogel particles, it is possible in one embodiment for the hydrogel particles to be placed in the first channel section as loose material, wherein the first channel section is constricted in such a way directly upstream and downstream of the loose material that the loose material remains in the place envisaged for it and forms the trap element. In another embodiment, the hydrogel particles can also be arranged in a multilayer construction between two bounding nets and in this way form the trap element.

In one embodiment, the trap element can be designed, for example, as bulk material or alternatively as pillars. The pillars can be designed as hydrogel structures or can be in the form of silica pillars or can be formed from other functionalized structures such as PMMA.

The trap element allows fluidic samples to pass through, the said samples comprising, for example, polynucleotides, proteins, lipids or hydrocarbons. According to the disclosure, the surfaces of the permeable element can be selected or modified in such a way that polynucleotides, proteins, lipids or hydrocarbons are preferentially retained. For example, filter membranes that consist of cellulose and can preferably be used for retaining nucleic acids are familiar to those skilled in the art.

In one embodiment, a closer element is arranged directly upstream of the trap element. “Directly” means that there are no further components, in particular no opener element, arranged between the trap element and the closer element.

### Receiving Region

Each channel section has precisely one receiving region, both the first and the n second, m third channel sections and f kth channel sections. The process media are supplied via the respective receiving regions.

In a preferred embodiment, each receiving region is configured in such a way that it can serve to dispense a sample or as a reservoir for process media or as a mixer structure.

A reservoir for process media can be made available, for example, by providing a blister containing the respective process medium. The blister is connected to the receiving region.

In addition, the receiving region can be designed in the form of a mixer structure. In this mixer structure, at least two process media are mixed and then pass into the associated channel section.

### Exhaust Valve

In one embodiment, all the channel sections apart from the first channel section have an exhaust valve.

Known exhaust valves are hydrophobic membranes. In a preferred embodiment the exhaust valve has a PTFE membrane or consists of the latter. The permeability of the PTFE membrane (polytetrafluoroethylene membrane) for aqueous solutions is dependent on the pressure in the channel section, and the hydrophilicity of the solution. As the pressure increases, the PTFE membrane becomes permeable for aqueous solutions.

In another embodiment of the disclosure, the exhaust valve is a closer element.

In another embodiment of the disclosure, the exhaust valve is a combination of a hydrophobic membrane and a closer element.

In one embodiment of the disclosure, each second, third and kth channel section is constructed as follows. At one end, the channel section has a receiving region, through which a fluid can be introduced into the channel section. The fluid flows through the channel section, which has an exhaust valve at its other end, thus enabling the air to escape from the channel section. Arranged directly upstream of the exhaust valve is a closer element, which is activated by the fluid contact and closes the channel section with respect to the exhaust valve, thus preventing any fluid from escaping from the exhaust valve. This embodiment is advantageous if use is made of a fluid which has such a low hydrophilicity that it can penetrate a hydrophobic membrane or if the pressure in the channel section is so high that the fluid penetrates the hydrophobic membrane.

In another embodiment of the disclosure, each second, third and kth channel section is constructed as follows. At one end, the channel section has a receiving region, through which a fluid can be introduced into the channel section. The fluid flows through the channel section, which has an exhaust valve at its other end, thus enabling the air to escape from the channel section. In this embodiment, the exhaust valve is a hydrophobic membrane, preferably a PTFE membrane. This embodiment is particularly advantageous if use is made of a fluid of which the hydrophilicity is sufficiently high to ensure that it cannot penetrate the hydrophobic membrane.

### Waste Chamber

In another embodiment of the present disclosure, the microfluidic apparatus furthermore has at least one waste chamber with an exhaust valve, wherein the first channel section opens into the waste chamber and wherein a closer element is arranged in the first channel section at the inlet of the waste chamber. The exhaust valve is arranged on the waste chamber in such a way that the air can escape from the first channel section through the exhaust valve. By means of the closer element at the inlet of the waste chamber, this chamber can be closed at a defined point in time since a fluid which is suitable for activating the closer element flows through the first channel section.

In another embodiment, the apparatus according to the disclosure has a plurality of waste chambers, wherein each further waste chamber after the first waste chamber can be connected fluidically to the first channel section by an opener element. The waste chambers are preferably arranged downstream of the trap element in the flow direction of a fluid if the apparatus has a trap element.

### Structure Support

In one embodiment of the disclosure, the channel sections, opener and closer elements, the trap region of the microfluidic apparatus and further elements, such as exhaust valves and waste chambers, are arranged in at least one structure support or are formed by the latter.

The structure support preferably has a material selected from the group comprising polyethylene (PE), polyethylene terephthalate (PET), polymethylmethacrylate (PMMA), cyclo-olefin copolymer (COC) and polycarbonate (PC). As a particular preference, the structure support is produced as a self-adhesive film consisting of one of these materials. In this embodiment, the microfluidic system furthermore comprises at least one cover, which at least partially covers the structure support.

The microfluidic apparatus according to the disclosure can be produced by the following production methods, for example:
laser structuring of the individual layers and joining by means of a hot press, adhesive films, for example;
soft lithography (production of a master (e.g. photoresist (e.g. SU8) or dry film resist (DFR)) and replication in, for example, polydimethylsiloxane (PDMS) or polyurethane (PU));
3D printing;
hot embossing;
lithography with wet/dry etching methods;
micro-machining (drilling and milling);
ultrasonic machining;
electro-erosive or electrochemical machining;
electron beam machining; and
focused ion beam machining.

The opener and closer elements by means of which control of the fluidic apparatus is implemented are advantageously activated by the fluids which function as process media in the fluidic system. This means that the fluidic sample or the fluids which are introduced as process media into the receiving regions of the channel sections directly control the activation of the opener and closer elements.

In another embodiment, the trap element is arranged as a connecting element between two structure supports.

In one embodiment of the present disclosure, the microfluidic apparatus furthermore has one or more pressure and/or flow sources, which provide the necessary fluid transport. The pressure and/or flow sources are preferably integrated directly into the fluidic apparatus or can be combined easily therewith. As a particular preference, the pressure and/or flow sources are embodied in such a way that they do not require any electrical energy for operation. Pressure and/or flow sources of this kind are known to those skilled in the art from DE102010015161, for example.

### Apparatus for Sample Analysis

In one embodiment, the fluidic connection according to the disclosure furthermore has an apparatus for sample analysis, which is arranged downstream of the trap region of the first channel section in the flow direction of a fluid, and is fluidically connected to the first channel section, preferably by at least one opener element. According to the disclosure, the apparatus for sample analysis can be arranged on the first channel section via one or more opener elements.

In a preferred embodiment, the apparatus for sample analysis is an apparatus for detecting analytes,
i. wherein the apparatus for sample analysis has j reaction chambers, wherein j∈□ and j≥1;
ii. wherein each reaction chamber has an inlet and an outlet; and
iii. wherein a closer element is arranged upstream of the inlet of each reaction chamber in the flow direction of a fluid, and an exhaust valve and/or closer element is arranged at the outlet of each reaction chamber.

In another embodiment, the apparatus for sample analysis is an apparatus for removing the isolated analyte in order then to feed it to other analyses or analyses to be carried out externally.

In one embodiment, the analytes to be detected are passed from the trap element into the apparatus for sample analysis in the elution solution after elution.

In one embodiment, dried reagents can be contained in one or more of the j reaction chambers. The reagents are preferably preserved by heat drying or lyophilization.

In one embodiment, the reagents are reagents for isothermal detection of nucleic acids. These can be, for example, loop-mediated isothermal amplification (LAMP), rolling circle amplification (RCA), recombinase polymerase amplification (RPA) or similar. Complex temperature protocols are thereby avoided.

The closer element at the inlet of each reaction chamber allows the dosing of the eluate and “sealing” of the chamber (with respect to adjacent reaction chambers and to the supply) in order to prevent cross contaminations. After complete filling of the reaction chamber, the closer elements at the inlet and outlet of each reaction chamber close the respective reaction chamber before the start of the biochemical detection reaction and thus prevent subsequent emergence of reaction solutions from the reaction chamber.

In one embodiment, the apparatus for sample analysis furthermore has/exhaust valves, wherein l∈□ and l≥1 and l≤j, wherein in each case precisely one exhaust valve is arranged in the outlet of a reaction chamber. The exhaust valve enables air to escape, while fluids are retained. In this way, it is possible to prevent air bubbles remaining before the closure of the reaction chambers. As a particular preference, the l exhaust valves are permeable membranes.

In a particularly preferred embodiment, the apparatus for sample analysis comprises two or more reaction chambers, which are arranged in such a way that the reaction chambers are filled uniformly, in parallel and at the same rate. Simultaneous detection (multiplex detection) of different analytes in an elution solution is thereby advantageously made possible.

In another embodiment, a mixing chamber, which enables thorough mixing of the eluate before the filling of the j reaction chambers, is arranged upstream of the j reaction chambers in order to compensate any possible elution gradient and thus allows filling of each of the j reaction chambers with the same concentration of analyte.

In another embodiment of the present disclosure, the fluidic apparatus according to the disclosure furthermore has a temperature control element. The apparatus for sample analysis preferably has a temperature control element, thereby enabling temperature control of the fluid to be performed. It is thus possible to control the temperature of fluids in the manner required for chemical analyses. Here, the temperature control element can be integrated into the fluidic apparatus or combined with the latter. An electric temperature control element is preferably used. However, the temperature control element can also be embodied in such a way that no electrical energy is required for its operation.

In one embodiment, the apparatus for sample analysis furthermore has at least one receiving region for a fluid. Process media can be fed to the apparatus for sample analysis through the at least one receiving region of the sample analyser. In another embodiment, the apparatus for sample analysis furthermore has cavities, which are in communication with a receiving region of the apparatus for sample analysis. In this embodiment, samples can be pipetted into the cavities. The filled cavities are then mechanically closed and/or sealed. Reagents and analytes can thus be stored in the apparatus for the automatic process sequence. The number of manual steps for sample analysis with the apparatus according to the disclosure is then reduced to
1. introduction of the sample into the receiving region of the first channel section when all other fluids (process media) are already in the receiving regions of the second, third and/or k^{th} channel sections and/or of the apparatus for sample analysis, or
2. introduction of the sample into the receiving region of the first channel section and activation of the apparatus by the introduction of the fluids (process media) into the receiving regions of the second, third and/or k^{th} channel sections and/or the apparatus for sample analysis of the fluidic apparatus.

### Method

The disclosure furthermore provides a method for the sequential supply of defined fluid volumes by means of a fluidic apparatus having a first channel section and at least one second channel section, at least one opener element and at least one closer element,
wherein
the at least one second channel section is arranged so as to adjoin the first channel section by means of an opener element, and the opener element is configured to establish a fluidic connection from the at least one second channel section to the first channel section;
the first and the at least one second channel section each have a receiving region,
a closer element is arranged in the first channel section upstream of the opener element in the flow direction of a fluid;
comprising the following steps:
a) supply of a first fluid in the receiving region of the first channel section, thereby forming a flow direction of the first fluid in the first channel section;
b) supply of a second fluid in a receiving region of an at least one second channel section, thereby forming a flow direction of the second fluid in the at least one second channel section;
c) activation of the closer element by the first fluid in the first channel section and flow of the first fluid through the first channel section until the closer element is closed;
d) activation of the at least one opener element by the first fluid in the first channel section and/or activation of the at least one opener element by the second fluid in the at least one second channel section and opening of a fluidic connection between the first and the at least one second channel section;
e) inflow of the second fluid from the at least one second channel section into the first channel section and flow through the first channel section in the flow direction of the first fluid.

All the features which have been described for the apparatus according to the disclosure apply in the same way to the method according to the disclosure and vice versa.

In a preferred embodiment, the fluidic apparatus according to the disclosure is configured to carry out the method according to the disclosure.

According to the method, a first fluid is provided in the receiving region of the first channel section. That is to say that the first fluid is introduced into the said channel section, thereby forming a flow direction of the first fluid in the first channel section. The first fluid can be of animal or human origin, for example, or alternatively a liquid containing components to be analysed. In one embodiment, the first fluid is a lysis and binding buffer containing components to be analysed.

According to the disclosure, a second fluid is provided in the receiving region of at least one second channel section. That is to say, the second fluid is introduced into the said channel section, thereby forming a flow direction of the second fluid in the at least one second channel section.

The first fluid flows through the first channel section and, in the process, activates the at least one closer element. The first fluid continues to flow through the first channel section until the closer element is closed. The volume of the first fluid which flows through the first channel section can thereby be precisely defined.

After contact between the first fluid and the at least one closer element, the opener element is activated by contact with the first fluid in the first channel section and/or activation of the opener element by the second fluid in the at least one second channel section. Once the at least one opener element is open, a fluidic connection is thereby established between the first and the at least one second channel section.

The second fluid can then flow out of the at least one second channel section into the first channel section and flow through the said first channel section in the same flow direction of the first fluid previously.

By means of the activation of the opener element, it is possible to precisely define the time sequence of the method. That is to say it is both possible to pass a defined volume of the first fluid through the first channel section at a defined time and to pass a defined volume of the second fluid through at a defined time.

In one embodiment of the method according to the disclosure, the fluidic apparatus furthermore has a trap element in the first channel section downstream of the opener element, through which the fluids flow. During the passage through the trap element, at least one component of the first fluid is retained and immobilized.

In another embodiment of the method according to the disclosure, the fluidic apparatus furthermore has an apparatus for sample analysis, which is arranged on the first channel section by means of an opener element, wherein the opener element is configured to establish a fluidic connection from the first channel section to the apparatus for sample analysis;
wherein the apparatus for sample analysis is arranged downstream, in the flow direction of a fluid, of the opener element which connects the second to the first channel section, or is arranged downstream of the trap element in the first channel section;
and wherein a fluid flow in the first channel section activates the opener element, which fluidically connects the apparatus for sample analysis to the first channel section.

In another embodiment of the method for the sequential supply of defined fluid volumes by means of a fluidic apparatus, the fluidic apparatus has
a first channel section and n second channel sections, wherein n∈□ and n≥1, at least one opener element and at least one closer element;
wherein
the first and the n second channel sections each have a receiving region;
each of the n second channel sections is arranged so as to adjoin the first channel section by means of an opener element, and each opener element is configured to establish a fluidic connection from a second channel section to the first channel section;
a respective closer element is arranged in the first channel section upstream of each opener element in the flow direction of a fluid;
comprising the following steps:
a. supply of a first fluid in the receiving region of the first channel section, thereby forming a flow direction of the first fluid in the first channel section;
b. flow of the first fluid through the first channel section, wherein the closer element between the receiving region of the first channel section and the first opener element in the flow direction of the first fluid is activated by the first fluid;
c. supply of a second fluid in the receiving region of a second channel section, which adjoins the first opener element, thereby forming a flow direction of the second fluid in the second channel section;
d. activation of the first opener element by the second fluid in the second channel section and/or by the first fluid in the first channel section and opening of a fluidic connection between the first and the second channel section;
e. inflow of the second fluid from the second channel section into the first channel section and flow through the first channel section;
f. activation of a closer element which is arranged between the last-activated opener element and a further opener element arranged in the flow direction of the first channel section until this closer element is closed by the fluid flowing through the first channel section;
g. supply of a further fluid in the receiving region of a further second channel section, which can be connected fluidically to the first channel section by the further opener element;
h. activation of the opener element of the further second channel section from method step g. by the fluid introduced into the further channel section in method step g. and/or by the fluid in the first channel section;
i. repetition of steps f. to h. until all the opener elements that are arranged upstream of the trap element in the flow direction in the first channel section are open.

In this way, it is possible, for example, to supply an elution buffer and a washing buffer. In this way, any number of further fluids can be fed to the first channel section in a defined volume with defined timing.

In one embodiment, fluids are fed to the first channel system 1, 2, 3, 4 or 5.

In another embodiment, the apparatus furthermore has m third channel sections. According to the disclosure, the m third channel sections are arranged in such a way that fluid can flow from them into a second channel section and from the second into the first channel section. By a suitable choice of opener and closer elements, both the volume of the individual fluids and the time sequence of the method steps can be precisely defined.

In another embodiment of the method for the sequential supply of defined fluid volumes by means of a fluidic apparatus, the fluidic apparatus has
a first channel section, a second channel section and m third channel sections, wherein m∈□ and m≥1, at least one opener element and at least one closer element;
wherein
the first, the second channel section and the m third channel sections each have a receiving region;
the second channel section adjoins the first channel section by means of an opener element, wherein the opener element is configured to establish a fluidic connection from the second channel section to the first channel section;
each of the m third channel sections adjoins the second channel section by means of a respective opener element, and each opener element is configured to establish a fluidic connection from a third channel section to the second channel section;
a closer element is arranged in the first channel section upstream of the opener element in the flow direction of a fluid;
comprising the following steps:
a. supply of a first fluid in the receiving region of the first channel section, thereby forming a flow direction of the first fluid in the first channel section;
b. flow of the first fluid through the first channel section, wherein the closer element between the receiving region of the first channel section and the first opener element in the flow direction of the first fluid is activated by the first fluid;
c. supply of a second fluid in the receiving region of the second channel section, which adjoins the first opener element, thereby forming a flow direction of the second fluid in the second channel section;
d. activation of the first opener element, which connects the first channel section and the second channel section, by the second fluid in the second channel section and/or by the first fluid in the first channel section and opening of a fluidic connection between the first and the second channel section;
e. inflow of the second fluid from the second channel section into the first channel section and flow through the first channel section;
f. supply of a further fluid in a receiving region of a third channel section, thereby forming a flow direction of the fluid in the third channel section;
g. activation of the opener element which connects the third channel section and the second channel section by the fluid in the second channel section and/or by the fluid in the third channel section and opening of a fluidic connection between the third and the second channel section;
h. inflow of the fluid from the third channel section into the second channel section and from the second channel section into the first channel section and flow through the first channel section;
i. supply of a further fluid in the receiving region of a further third channel section, which can be connected fluidically to the second channel section by a further opener element;
j. activation of the opener element of the further third channel section from method step i. by the fluid introduced into the further third channel section in method step i. and/or by the fluid in the second channel section;
k. inflow of the fluid from the further third channel section into the second channel section and from the second channel section into the first channel section and flow through the first channel section;
l. repetition of steps i. to k. until all the opener elements that connect a third channel section to the second channel section are open.

In another embodiment, the apparatus has not only m third channel sections but also ƒ k^{th} channel sections. According to the disclosure, the ƒ k^{th} channel sections are each arranged so as to adjoin at least one (k−1)^{th} channel section by means of a respective opener element. A fluid can therefore be carried from a k^{th} channel section via a (k−1)^{th} channel section until it enters a third channel section and, from the latter, a second and then the first channel section.

In another embodiment of the method for the sequential supply of defined fluid volumes by means of a fluidic apparatus, the fluidic apparatus has
a first channel section, a second channel section, a third and ƒ k^{th} channel sections, wherein ƒ∈□ and ƒ≥1 and k∈□ and k≥4, at least one opener element and at least one closer element;
wherein
the first, the second channel section, the third and the ƒ k^{th} channel sections each have a receiving region;
the second channel section adjoins the first channel section by means of an opener element, wherein the opener element is configured to establish a fluidic connection from the second channel section to the first channel section;
the third channel section adjoins the second channel section by means of an opener element, wherein the opener element is configured to establish a fluidic connection from the third channel section to the second channel section;
each of the ƒ k^{th} channel sections adjoins a (k−1)^{th} channel section by means of an opener element and each opener element is configured to establish a fluidic connection from a k^{th} channel section to the (k−1)^{th} channel section;
each of the ƒ k^{th} channel sections, where k=4, adjoins the third channel section by means of a respective opener element, and each opener element is configured to establish a fluidic connection from the k^{th} channel section to the third channel section;
a closer element is arranged in the first channel section upstream of the opener element in the flow direction of a fluid;
comprising the following steps:
a. supply of a first fluid in the receiving region of the first channel section, thereby forming a flow direction of the first fluid in the first channel section;
b. flow of the first fluid through the first channel section, wherein the closer element between the receiving region of the first channel section and the first opener element in the flow direction of the first fluid is activated by the first fluid;
c. supply of a second fluid in the receiving region of the second channel section, which adjoins the first opener element, thereby forming a flow direction of the second fluid in the second channel section;
d. activation of the first opener element, which connects the first channel section and the second channel section, by the second fluid in the second channel section and/or by the first fluid in the first channel section and opening of a fluidic connection between the first and the second channel section;
e. inflow of the second fluid from the second channel section into the first channel section and flow through the first channel section;
f. supply of a fluid in the receiving region of the third channel section, thereby forming a flow direction of the fluid in the third channel section;
g. activation of the opener element which connects the third channel section and the second channel section by the fluid in the second channel section and/or by the fluid in the third channel section and opening of a fluidic connection between the third and the second channel section;
h. inflow of the fluid from the third channel section into the second channel section and from the second channel section into the first channel section and flow through the first channel section;
i. supply of a fluid in the receiving region of a k^{th} channel section, thereby forming a flow direction of the fluid in the k^{th} channel section;
j. activation of the opener element which connects the k^{th} channel section and the (k−1)^{th} channel section by the fluid in the k^{th} channel section and/or by the fluid in the (k−1)^{th} channel section and opening of a fluidic connection between the k^{th} channel section and the (k−1)^{th} channel section;
k. inflow of the fluid from the k^{th} channel section into the (k−1)^{th} channel section and flow through the channel sections as far as the first channel section;
l. supply of a further fluid in the receiving region of a further k^{th} channel section, thereby forming a flow direction of the fluid in the further k^{th} channel section;
m. activation of the opener element which connects the further k^{th} channel section and the (k−1)^{th} channel section by the fluid in the k^{th} channel section and/or by the fluid in the (k−1)^{th} channel section and opening of a fluidic connection between the k^{th} and the (k−1)^{th} channel section;
n. inflow of the fluid from the k^{th} channel section into the (k−1)^{th} channel section and flow through the channel sections as far as the first channel section;
o. repetition of steps l. to n. until all the opener elements that connect a k^{th} channel section fluidically to a (k−1)^{th} channel section are open.

In another embodiment, the fluidic apparatus furthermore has a trap element, an apparatus for sample analysis and a waste chamber having an exhaust valve.

In another embodiment of the method for the sequential supply of defined fluid volumes by means of a fluidic apparatus, the fluidic apparatus has
a first channel section and n second channel sections, wherein n∈□ and n≥1, at least one opener element and at least one closer element;
a trap element;
an apparatus for sample analysis; and
a waste chamber having an exhaust valve;
wherein
the first and the n second channel sections each have a receiving region;
each of the n second channel sections is arranged so as to adjoin the first channel section by means of a respective opener element, and each opener element is configured to establish a fluidic connection from a second channel section to the first channel section;
a respective closer element is arranged in the first channel section upstream of each opener element in the flow direction of a fluid;
the trap element is arranged in the first channel section downstream of the last opener element in the flow direction of a fluid;
the apparatus for sample analysis is arranged in the first channel section downstream of the trap element in the flow direction of a fluid and can be connected fluidically to the said channel section by at least one opener element; and
the first channel section opens into the waste chamber, wherein a closer element is arranged in the first channel section at the inlet of the waste chamber;
comprising the following steps:
a. supply of a first fluid in the receiving region of the first channel section, thereby forming a flow direction of the first fluid in the first channel section;
b. flow of the first fluid through the first channel section and the trap element arranged therein to the waste chamber, wherein the closer element between the receiving region of the first channel section and the first opener element in the flow direction of the first fluid is activated by the first fluid, and the closer element upstream of the waste chamber;
c. supply of a second fluid in the receiving region of a second channel section, which adjoins the first opener element, thereby forming a flow direction of the second fluid in the second channel section;
d. activation of the first opener element by the second fluid in the second channel section and/or by the first fluid in the first channel section and opening of a fluidic connection between the first and the second channel section;
e. inflow of the second fluid from the second channel section into the first channel section and flow through the first channel section and the trap element arranged therein;
f. activation of at least one opener element which connects the apparatus for sample analysis fluidically to the first channel section by the fluid flowing through the first channel section;
g. inflow of the fluid from the first channel section into the apparatus for sample analysis.

According to the method, a first fluid is supplied in the receiving region of the first channel section. That is to say that the first fluid is introduced into the said channel section, thereby forming a flow direction of the first fluid in the first channel section. The first fluid flows through the first channel section and the trap element arranged therein as far as the waste chamber. During passage through the trap element, at least one component of the first fluid is retained and immobilized. The first fluid activates the closer element between the receiving region of the first channel section and the first opener element in the flow direction of the first fluid. The flow of the first fluid from the receiving region through the first channel section is thereby blocked, as a result of which no more fluid can flow from the receiving region to the trap element and thus dosing of the first fluid is performed. In addition, the closer element upstream of the waste chamber is activated, and therefore this closer element is closed after a defined time.

A second fluid is introduced into the receiving region of a second channel section, which adjoins the first opener element. According to the disclosure, the first opener element is activated by the second fluid in the second channel section and/or by the first fluid in the first channel section, and therefore there is a fluidic connection between the first and the second channel section after a defined time. As a result, the second fluid passes out of the second channel section into the first channel section, flows through the latter in the direction of the trap element and flows through the trap element. According to the disclosure, the fluid in the first channel section activates at least one opener element which connects the apparatus for sample analysis fluidically to the first channel section and, if the at least one opener element is open, flows out of the first channel section into the apparatus for sample analysis. According to the disclosure, the time sequence of the method is determined by the activation of the opener and closer elements. It is also determined, in particular, by the structural arrangement of the elements of the apparatus.

The fluid which is introduced in method step c. can be a washing buffer, for example.

The method according to the disclosure is advantageous, in particular, for the time control of a sequential process sequence involving at least two liquids in a fluidic apparatus. By suitable selection of the parameters, such as opener and closer elements, the respectively desired time sequence of process steps, such as dosing, immobilization, washing, elution, dissolution of barriers and sealing of desired channel sections by means of closer elements can be achieved in accordance with the process.

In one embodiment of the present disclosure, a further closer element is arranged in the first channel section downstream of the first opener element and upstream of the trap region. This is preferably activated by the fluid which is introduced through the further channel section in step c. The closer element preferably closes the first channel section when a defined volume of the fluid has flowed through. This embodiment is used for volume definition of the fluid.

In another embodiment, the method according to the disclosure furthermore has the following steps after the method step e. just described and before the method step f. just described:
ea. activation of a closer element which is arranged between the just activated opener element and a further opener element arranged in the flow direction of the first channel section until the said closer element is closed by the fluid flowing through the first channel section;
eb. supply of a further fluid in the receiving region of a further second channel section, which can be connected fluidically to the first channel section by the further opener element;
ec. activation of the opener element of the further second channel section from method step eb. by the fluid introduced into the further channel section in method step eb. and/or by the fluid in the first channel section;
ed. repetition of steps ea. to ec. until all the opener elements which are arranged upstream of the trap element in the flow direction in the first channel section are open.

In another embodiment, the apparatus furthermore has m third channel sections. According to the disclosure, the m third channel sections are arranged in such a way, as already described, that a fluid can flow from them into a second channel section and, from the second channel section, into the first channel section. By suitable selection of the opener and closer elements, both the volume of the individual fluids and the time sequence of the method steps can be precisely defined.

In another embodiment, the apparatus has not only m third channel sections but also ƒ k^{th} channel sections. According to the disclosure, the ƒ k^{th} channel sections are each arranged so as to adjoin at least one (k−1)^{th} channel sections by means of an opener element. It is therefore possible to pass a fluid from a k^{th} channel section via a (k−1)^{th} channel section, as already described, until it gets into a third channel section and, from the latter, into a second channel section and then into the first channel section.

If the apparatus has m third channel sections or additionally ƒ k^{th} channel sections, it is likewise possible in the same way, as already described, for there to be a trap element, an apparatus for sample analysis and a waste chamber having an exhaust valve in the apparatus.

In one embodiment, the opener element which connects the first channel section to the apparatus for sample analysis is already activated by contact with the fluid which is introduced into the receiving region of the first channel section, i.e. the activation of the opener element is initiated. The activation time is then matched to the method sequence. In one embodiment, the activation of this opener element can therefore be initiated by the fluid which is introduced into the receiving region of the first channel section, and the activation time is complete when the last fluid flows out of a second, third or k^{th} channel section through the first channel section. This fluid can then flow into the apparatus for sample analysis through the open opener valve.

The present disclosure can be used as a chemofluidic system or chemofluidic apparatus for the preparation of samples for further analyses, e.g. on the basis of nucleic acids, proteins, lipids and/or carbohydrates. Analyses can comprise processes on the basis of polymerase chain reactions, isothermal amplification, detection of enzyme activities and/or other biochemical detection reactions.

According to the disclosure, the apparatus for sample analysis can be configured in many different ways and is configured according to the respective application. In one embodiment, the apparatus for sample analysis has opener elements and/or closer elements according to the present disclosure.

One major advantage of the present disclosure consists in that, by virtue of integration of a trap element into a chemofluidic system, at least one component of a sample can be retained/immobilized by a skilful combination of opener elements and closer elements. Thus, the apparatus according to the disclosure, as a chemofluidic system, allows the preparation of a sample of animal or human origin for subsequent analysis, e.g. by means of isothermal amplification, by automation of the complex process sequence without auxiliary energy.

The present disclosure enables automated execution, without auxiliary energy, of complex biochemical process sequences of the kind that are frequently encountered in the preparation of biological samples for subsequent analysis. Through the combination of closer and opener elements, control of the process sequence in the microfluidic system—activated by the process media-is achieved, and external control becomes unnecessary. This also confers advantages in the domain of parallelization and miniaturization. In the case of the closer elements, it is possible to successfully photostructure elements from 50 μm and, in the case of the opener elements, this is possible from a hole diameter (hole design) of from 125 μm. Scaling of the channel sections at these orders of magnitude is possible.

Through the integration of a trap element, it is possible to selectively retain/immobilise components of a sample in order to make them available to a subsequent analysis. Through immobilization and washing steps following binding, it is possible to remove components of the sample which have a disruptive effect on the detection reactions, without losing those components of the sample that are to be analysed.

In a further embodiment, the first channel section of the present disclosure has a mixer structure as a receiving region. A fluidic sample, e.g. a patient sample, is introduced into a channel structure upstream of the mixer structure via a receiving region, and a further fluidic sample, e.g. a lysis/binding buffer, is introduced via a further receiving region. The two fluidic samples are fed in parallel into the mixer structure, which acts as the receiving region of the first channel section. The volumes of both fluidic samples are predefined and the two fluidic samples are mixed in the mixer structure. This mixture continues to flow through the first channel section via a trap element, which can be a permeable membrane, into a waste chamber. The air present in the channel structure can escape through an exhaust valve placed in the waste chamber, whereby no liquid can escape. The mixture of fluidic sample and other fluidic sample activates opener elements and closer elements on its way from the mixer structure to the waste chamber.

A first buffer, e.g. a wash buffer, is added to the channel structure support via a receiving region of a second channel section. The buffer flows through the second channel section to a permeable membrane, which allows air, but not liquid, to escape from the channel structure. In this embodiment, the permeable membrane is part of an exhaust valve in the second channel section. The buffer activates an opener element adjacent to the first channel section. After a defined time, this opener element opens so that the buffer can flow from the second channel section into the first channel section in the direction of the trap element. The buffer flows over the trap element and can remove non-immobilised components from the trap element. In this embodiment, the volume of the second buffer is defined by the size of the waste chamber behind the trap element in the first channel section. Once the waste chamber is filled with the mixed fluidic samples and the buffer, the inflow of buffer stops.

A second buffer, e.g. an elution buffer, can be applied to the channel structure support via a receiving region in a further second channel section. The second buffer flows in the further second channel section to a permeable membrane, which allows air, but not liquid, to escape from the channel structure. In this embodiment, the permeable membrane is part of an exhaust valve in the second channel section. The second buffer activates an opener element adjacent to the first channel section. After the inflow of the first buffer, e.g. wash buffer, is stopped, the opener element between the first channel section and the further second channel section opens and allows the second buffer to flow from the further second channel section into the first channel section in the direction of the reaction chambers, which are part of a sample analyser. The second buffer also flows over the trap element and can detach the components of the fluid sample immobilised there.

The reaction chambers, which are part of the sample analyser, are connected to the first channel section by an opener element. When this opener element opens after the inflow of the second buffer, e.g. elution buffer, the flow path for the second buffer, e.g. elution buffer with components detached from the trap element, is released into the reaction chambers belonging to the sample analyser. The buffer can flow in the direction of the reaction chambers, whereby a closer element is arranged before each reaction chamber and after each reaction chamber in order to define the volume of liquid in the reaction chamber and immediately close the flow paths. The closer elements are supported by downstream permeable membranes and only allow air, not liquid, to escape.

The present disclosure is furthermore explained in greater detail below with reference to 15 figures and 4 exemplary embodiments.

### BRIEF DESCRIPTION OF THE FIGURES

FIGS. 1A and B illustrate an opener element;

FIG. 2 illustrates an embodiment of the opener element;

FIGS. 3A and B illustrate a closer element;

FIGS. 4A and B illustrate trap elements;

FIGS. 5A, B, C, and D illustrate embodiments of the trap element;

FIG. 6 illustrates an embodiment of the apparatus according to the disclosure;

FIGS. 7A and B illustrate embodiments of the present disclosure;

FIGS. 8A and B illustrate embodiments of the present disclosure;

FIG. 9 illustrates an embodiment of the present disclosure;

FIGS. 10A and B illustrate an embodiment of the present disclosure;

FIG. 11 illustrates an embodiment of the present disclosure;

FIG. 12 illustrates an embodiment of the present disclosure;

FIGS. 13A and B illustrate opening times for various opener elements;

FIGS. 14A and B illustrate closing times for various closer elements;

FIG. 15 embodiments of mixer structures/receiving region.

### DETAILED DESCRIPTION

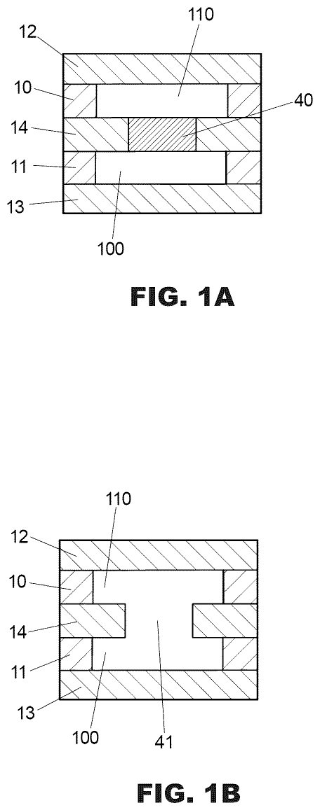

FIGS. 1A and B illustrate the basic structure and operation of a time- and event-controlled opener element 40. FIG. 1A illustrates the opener element (40) in the closed state, and FIG. 1B illustrates it in the open state.

The opener element 40 separates a first channel section 100 in the structure support 11 and a second channel section 110 in the structure support 10. When a process medium impinges upon the opener element 40 in the structure support 14 through one of the channel sections 100, 110, it initiates the dissolution process of the said opener. After a defined time (determined by the design, material etc.), the material of the opener element 40 has completely dissolved, and a connection 41 between the first channel section 100 and the second channel section 110 has been created. The structure supports 12 and 13 each form a cover.

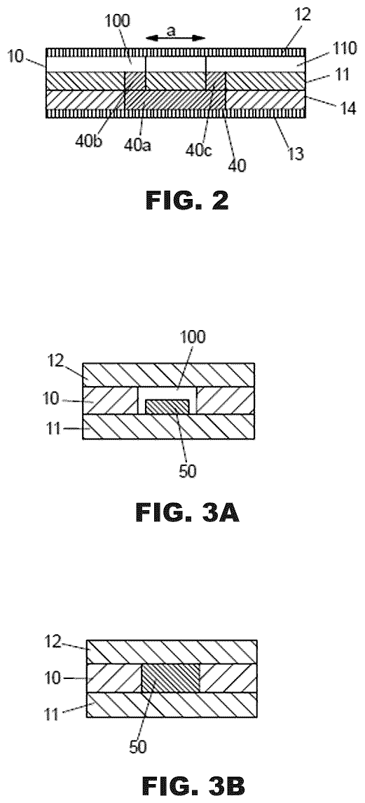

FIG. 2 illustrates another embodiment of the opener element 40. In this embodiment, a first channel section 100 and a second channel section 110 are arranged peripherally with respect to one another. The opener element 40 is arranged in the structure supports 11 and 14, which are arranged on the structure support 10 that has the channel sections 100 and 110. Here, the opener element 40 overlaps with channel section 100 and channel section 110. The opener element 40 has a channel-shaped part 40a and two connecting elements 40b, 40c. When a process medium impinges upon the opener element 40 through one of the channel sections 100, 110 or through both channel sections, it initiates the dissolution process of the said opener. After a defined time (determined by the design, material etc.), the material of the opener element 40 has completely dissolved, and a connection between the first channel section 100 and the second channel section 110 has been created. The step width of the opener element 40 is indicated by the reference sign a.

FIGS. 3A and B illustrate the basic structure and operation of a time-and event-controlled closer element 50. The closer element is mounted on structure support 11, and the channel section 100 is introduced into structure support 10. Structure support 12 forms a cover. By way of example, the figure illustrates a closer element which is actuated first of all in an event-controlled manner and then in a time-controlled manner. The purpose of the closer element is to close the channel section 100 after filling with process medium, the volume of the process medium being defined by the closing process. When a process medium impinges upon the closer element 50 in the dry state through the channel section 100 (FIG. 2A), it initiates the swelling of the said closer element. After a defined time (determined by the design, material etc.), the closer element 50 has swollen fully and closes the channel section 100 (FIG. 3B).

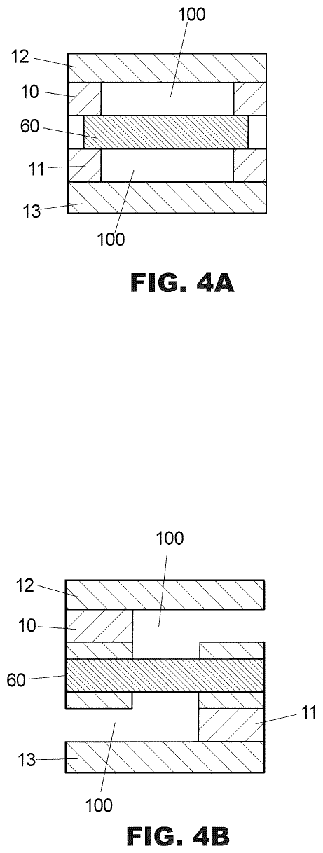

FIGS. 4A and B illustrate the basic structure of trap element 60. FIG. 4A illustrates a cross section through a channel section 100 with a permeable membrane as a trap element 60. FIG. 4 B illustrates a cross section in the longitudinal direction through a channel section 100 with a permeable membrane as a trap element 60.

These figures illustrate, by way of example, the integration of a structure support comprising a permeable membrane as a trap element 60. The trap element 60 is clamped between two layers 10, 11, which allow direct contact with the permeable membrane of the trap element 60 in the region of the channel section 100. In this case, the trap element 60 can be interpreted as a structure support per se. In one embodiment, the trap element is in the form of a film. Laser-cut into this is a hole, into which a cellulose or silica membrane is inserted. Thus, a process medium flows from one part of the channel section 100, which is arranged in a first structure support 10, through the trap element 60 into a further part of the channel section 100, which is arranged in a second structure support 11. In this case, the permeable membrane of the trap element 60 retains and immobilizes components of the process medium or sample.

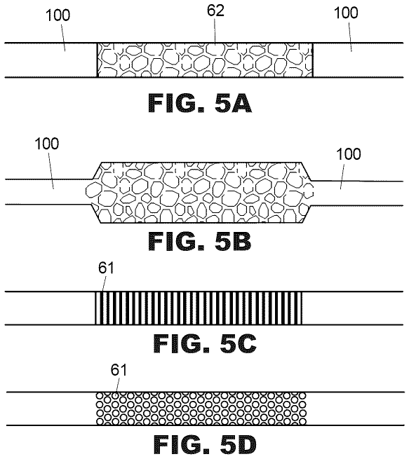

FIG. 5 illustrates various embodiments of the trap element in the form of integrated granules, e.g. hydrogel particles or functionalized silica particles. FIG. 5A illustrates the fixing of the granules at a defined position of a channel section 100 by means of a grid 62. FIG. 5 B illustrates fixing of the granules by means of a constriction of the channel section 100. In another embodiment, the trap element can be in the form of pillars which make available the largest possible surface area to the fluid. The pillars can be designed as hydrogel structures or be in the form of silica pillars or can be formed from other functionalized structures (e.g. PMMA). FIG. 5C shows a pillar structure 61 in side view, and FIG. 5D shows it in plan view.

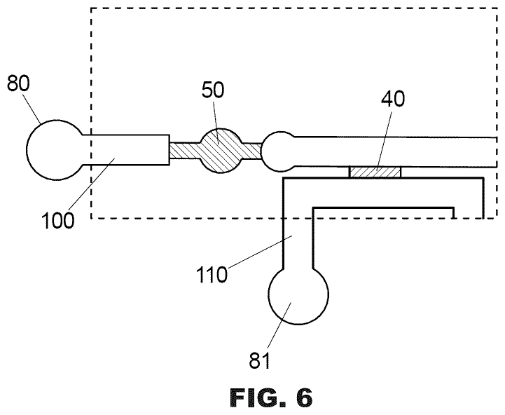

FIG. 6 illustrates the basic structure of the apparatus according to the disclosure. The apparatus according to the disclosure has a first channel section 100 and a second channel section 110, which are arranged peripherally with respect to one another. Here, the channel section can be fluidically connected to the first channel section via an opener element 40. In addition, a closer element 50 is arranged in the first channel section 100 upstream of the opener element 40. The first channel section 100 has a receiving region 80, and the second channel section has a receiving region 81.

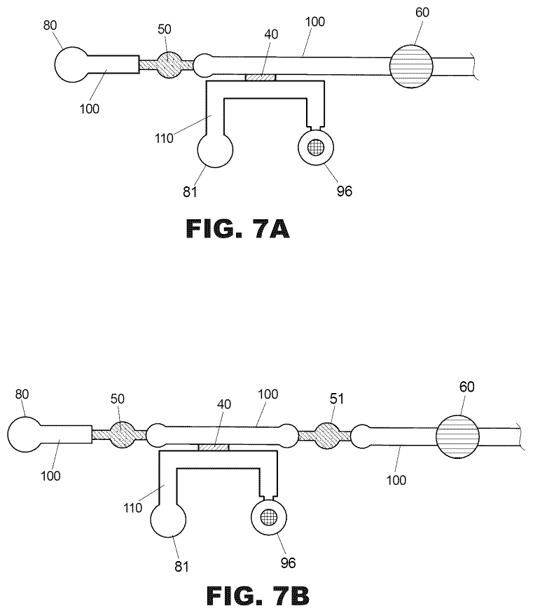

FIGS. 7A and B illustrate embodiments of the present disclosure. FIG. 7A illustrates a first channel section 100 with a receiving region 80. A fluid sample is introduced into the receiving region 80 and flows through the first channel section 100 to the trap element 60 and through the latter. As it flows through the first channel section 100, the closer element 50 is activated by the fluid sample and closes the channel section 100 with respect to the receiving region 80 after a defined time. It is thus not possible for another sample to flow in behind from the receiving region 80, as a result of which the volume of the fluid sample that passes through the trap element 60 is defined.

A liquid is likewise introduced into the receiving region 81 of a second channel section 110 and flows through the second channel section 110 to the exhaust valve 96. The exhaust valve 96 enables the air to escape from the channel section 110, but the exhaust valve 96 is configured in such a way that no liquid escapes. The opener element 40 is activated by the fluid sample in the first channel section 100 and/or by the liquid in the second channel section 110. The barrier of the opener element 40 dissolves until there is a fluidic connection between the first channel section 100 and the second channel section 110, thus enabling the liquid to flow out of the second channel section 110 into the first channel section 100 and then to flow through the trap element 60 in the said first channel section.

FIG. 7B illustrates a similar structure to FIG. 7A but, in this arrangement, another closer element 51 is provided in the first channel section 100, being arranged downstream of the opener element 40. The closer element 51 is activated by the liquid from the second channel section 110 and/or by the fluid sample that has flowed through before when the latter was flowing to the trap element 60. After a defined time, the closer element 51 blocks the first channel section 100, and no more liquid can flow from the second channel section 110 to the trap element 60, thereby defining the volume of liquid from the second channel section 110 which passes through the trap element 60.

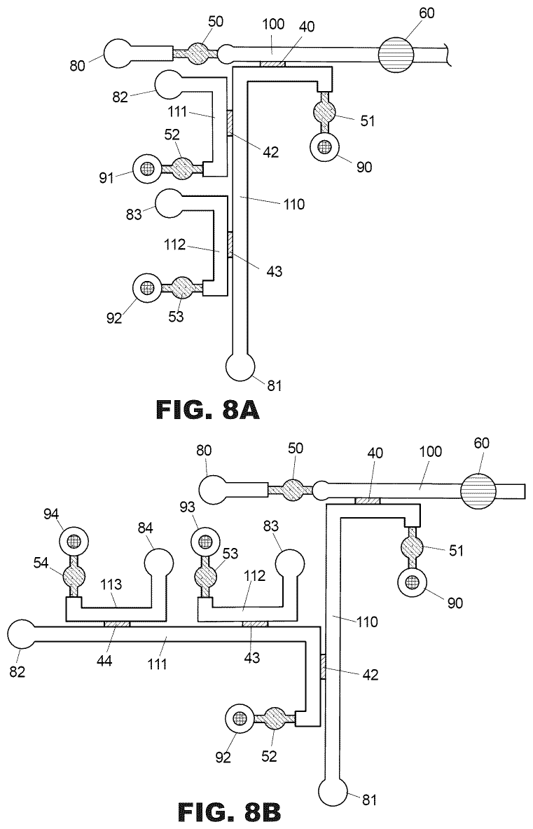

FIG. 8A illustrates an embodiment of the apparatus according to the disclosure. The first channel section 100 has a receiving region 80. A fluid sample is introduced into the receiving region 80 and flows through the first channel section 100 to the trap element 60 and through the latter. A second channel section 110 is arranged peripherally with respect to the first channel section 100. The first channel section 100 and the second channel section 110 can be connected fluidically by a first opener element 40. Two third channel sections 111, 112 are arranged peripherally with respect to the second channel section 110 and can be connected fluidically to the second channel section 110 by respective opener elements 42, 43. When a fluid sample or additional liquids are introduced into the receiving regions 80-83 of the first and second channel sections and/or the third channel section, the dissolution process of the respective opener elements adjoining these channel sections is started. After a defined time, the fluidic connection between the first channel section 100 and the second channel section 110 is thereby established, and the liquid flows out of the second channel section 110 to/through the trap element 60. After a defined time, the opener elements 42, 43 then open and thus allow a sequential flow of process medium to or through the trap element 60.

FIG. 8B illustrates another embodiment of the apparatus according to the disclosure. In this embodiment, the apparatus according to the disclosure has a first channel section 100, a second channel section 110, a third channel section 111 and two fourth channel sections 112, 113. Here, the second channel section 110 can be connected fluidically to the first channel section 100 via the opener element 40. The third channel section 111, in turn, can be connected fluidically to the second channel section 110 via the opener element 42. The two fourth channel sections 112 and 113 can be connected fluidically to the third channel section 111 via the opener elements 43, 44. Furthermore, all the features already described apply equally to this embodiment.

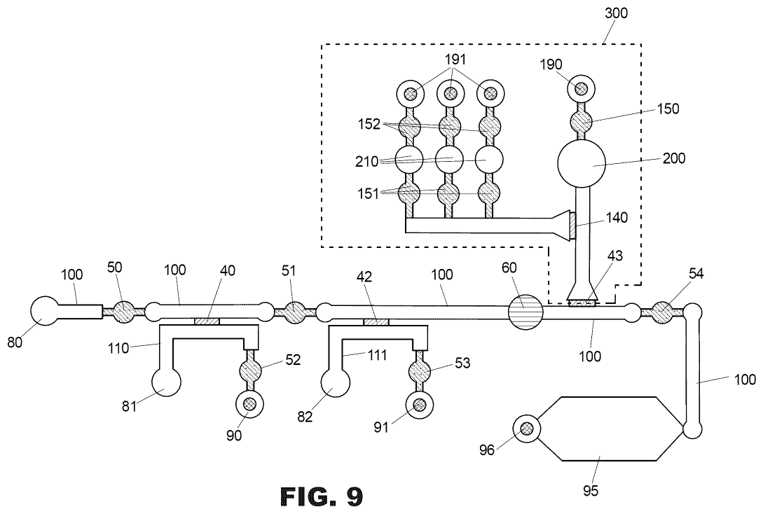

FIG. 9 illustrates an embodiment of the present disclosure which forms a chemofluidic system for the preparation of a sample with subsequent detection of the components. The illustrated circuit diagram of an LOC channel structure is suitable for the preparation of, for example, biological samples for subsequent processing, e.g. the detection of nucleic acids by means of isothermal amplification.

Via the receiving region 80, a fluidic sample can be applied to the first channel section 100. This sample flows through the first channel section 100, via the trap element 60, which can be a permeable membrane, into the waste chamber 95. The air present in the channel structure can escape through an exhaust valve 96 placed in the waste chamber 95, while no liquid can escape. On its way from the receiving region 80 to the waste chamber 95, the fluidic sample activates the opener elements 40, 42, 43 and the closer elements 50, 51, 54. Closer element 50 closes the first channel section with respect to the receiving region 80 after a predefined time and thus defines the sample volume which flows through the first channel section 100.

Via receiving region 81, a buffer, e.g. a washing buffer, is applied to the channel structure support. The buffer flows through the second channel section 110 to the permeable membrane 90, which allows air but no liquid to escape from the channel structure. The permeable membrane 90 is part of an exhaust valve. An outlet opening can furthermore be arranged at the permeable membrane 90 in order to enable cleaning of the device. The buffer additionally activates the opener element 40 from the side of the second channel section 110 and likewise the closer element 52. The closer element 52 supports the permeable membrane 90 and stops the liquid flow by closing the flow path after a predefined time. Once the closer element 50 has closed the flow path, the opener element 40 opens and allows the buffer to flow from the second channel section 110 into the first channel section 100. The buffer flows via the trap element 60 and can remove components that have not been immobilized from the permeable membrane. The volume of the first buffer is defined by way of the size of the waste chamber 95 and by the closer elements 51 and 54. If at least one of the elements closes the flow path, the addition of a first buffer stops.

Via the receiving region 82, a second buffer, e.g. an elution buffer, can be applied to the channel structure support. This flows in a third channel section 111 to the permeable membrane 91, which allows air but no liquid to escape from the channel structure. The permeable membrane 91 is part of an exhaust valve. An outlet opening can furthermore be arranged at the permeable membrane 91 in order to enable cleaning of the device. The second buffer additionally activates the opener element 42 and furthermore the closer element 53. The closer element 53 is part of the exhaust valve and supports the permeable membrane 91 and stops the liquid flow by closing the flow path after a predefined time. Once the closer elements 51 and 54 have closed the flow path, the opener element 42 opens and allows the second buffer to flow from the further second channel section 111 into the first channel section 100 in the direction of the chamber 200. The chamber 200 belongs to the apparatus for sample analysis 300, which is connected to the first channel section 100 by the opener element 43

The second buffer likewise flows via the trap element 60 and can remove those components of the fluid sample that have been immobilized there. The chamber 200 has a closer element 150 and a permeable membrane 190, which jointly form an exhaust valve. The closer element 150 is activated by the fluid flow and immediately closes the channel in order-together with the permeable membrane 190-to allow only the air but not the liquid to escape from the flow path. On its way to the chamber 200, the liquid activates the opener element 140. After a predefined time, this opener element 140 opens the flow path in the direction of the reaction chambers 210, wherein one closer element 151 is arranged upstream of each reaction chamber and one closer element 152 is arranged downstream of each reaction chamber in order to define the volume of the liquid in the reaction chamber 210 and to immediately close the flow paths. In this case, the closer elements 152 are supported by downstream permeable membranes 191 and allow only air and not liquid to escape.

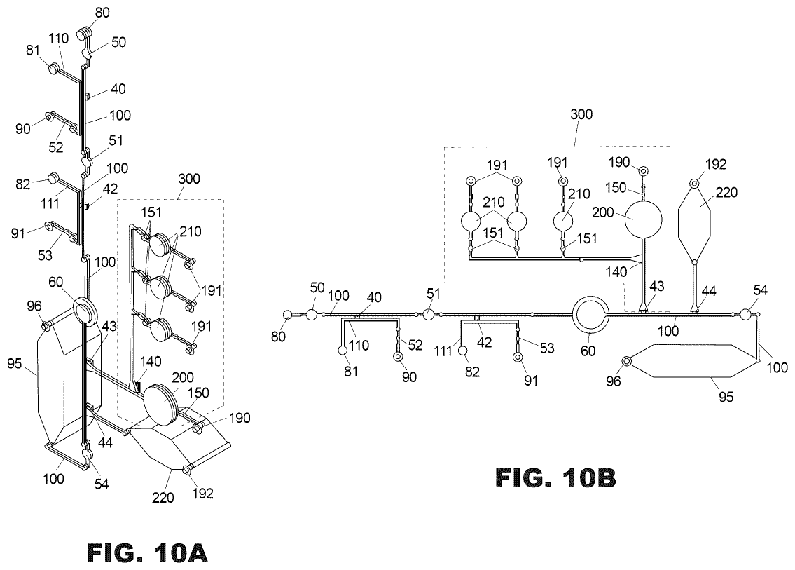

FIGS. 10A and B illustrate another embodiment of the present disclosure. FIG. 10 A shows the embodiment in a 3D illustration, while FIG. 10 B shows the embodiment in plan view. Fundamentally, the construction of the apparatus according to the disclosure corresponds to the construction shown in FIG. 9. In this embodiment, two waste chambers 95, 220 are arranged downstream of the trap element 60, wherein second waste chamber 220 is connected via the opener element 44 to the first channel section. This opens after the filling of the waste chamber 95 and the closure of the closer element 54. The second waste chamber 220 has a downstream exhaust valve 192. The apparatus for sample analysis 300 is arranged on the first channel section via the opener element 43. The apparatus is built up in structure layers.

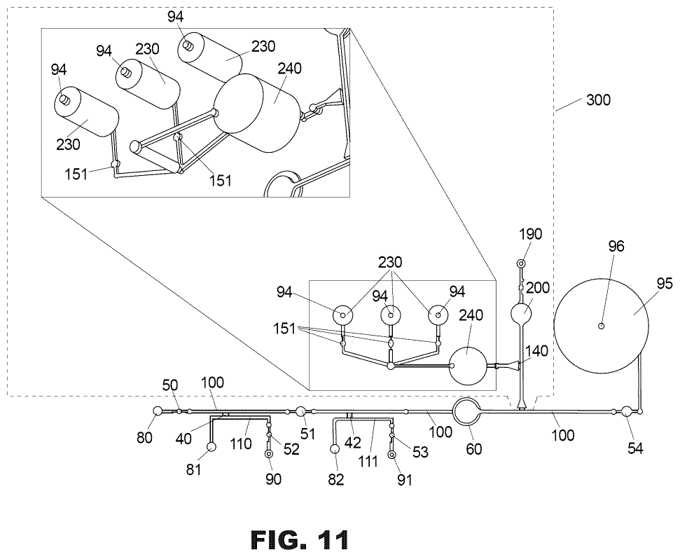

FIG. 11 illustrates an embodiment of the present disclosure comprising an apparatus for sample analysis 300. Fundamentally, the construction of the apparatus according to the disclosure corresponds to the construction shown in FIG. 9. In this embodiment, a mixing chamber 240 is arranged upstream of the three reaction chambers 230 (height: 4 mm, diameter 2.2 mm; volume 15 μl in each case). This allows thorough mixing of the eluate before the filling of the reaction chambers 230 in order to compensate for any possible elution gradient and allows the filling of each reaction chamber 230 with the same concentration of the analyte. Each reaction chamber 230 has a closer element 151 in the inlet region as well as a downstream permeable membrane.

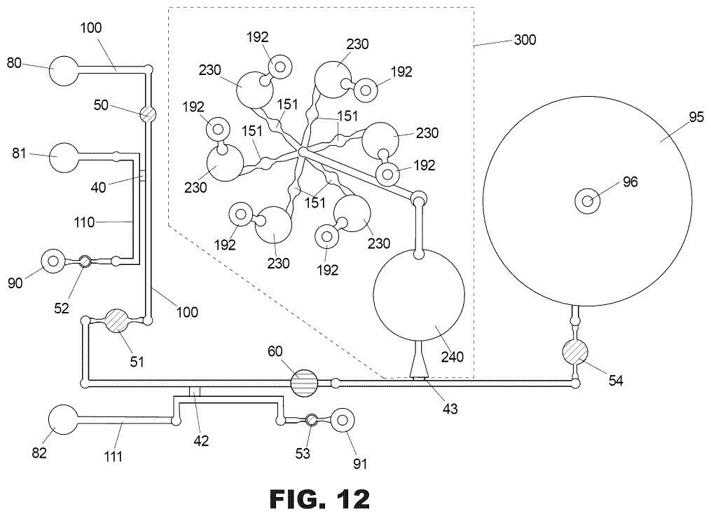

FIG. 12 illustrates another embodiment of the present disclosure comprising a sample analyser 300. Fundamentally, the construction of the apparatus according to the disclosure corresponds to the construction shown in FIG. 11. In this embodiment, six reaction chambers (height 4 mm in each case; diameter 1.75 mm in each case; volume 10 μl in each case) are arranged in such a way that uniform and parallel filling of these chambers with the eluate thoroughly mixed in the mixing chamber 240 (height: 4 mm, diameter 4.4 mm, volume: 60 μl) takes place. Each reaction chamber 230 has a closer element 151 in the inlet region as well as a downstream exhaust valve 192.

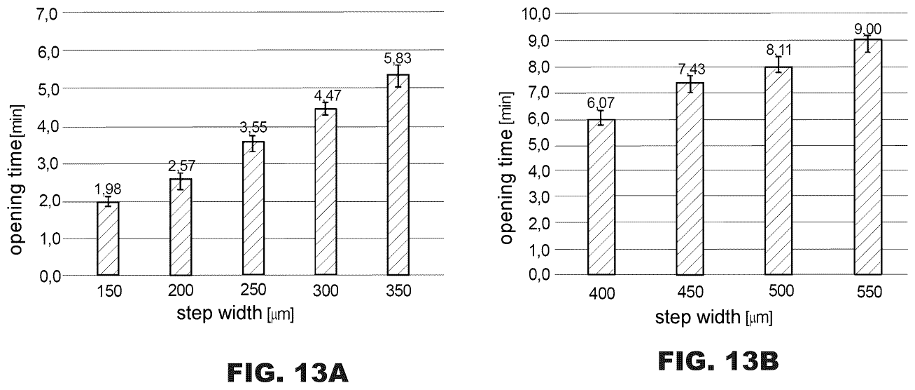

FIGS. 13A and B illustrate opening times for various opener elements 40. To simulate an extraction process, a lysis/binding buffer flowed through the first channel section for two minutes, and a washing buffer flowed through a further channel section for 10 seconds. The first channel section and the further channel section were connected by an opener element consisting of polyethylene glycol (MW 1 kDa and MW 1.5 kDa in a ratio of 2:10). The test was carried out for various step widths of the opener element. It can be clearly seen in FIG. 13 A how the opening time of the opener element depends on the step width with test parameters that are otherwise the same. The pressure was 150 mbar at a flow rate of 100 μl/min, and the test was carried out 3 times for each size of opener element 3.

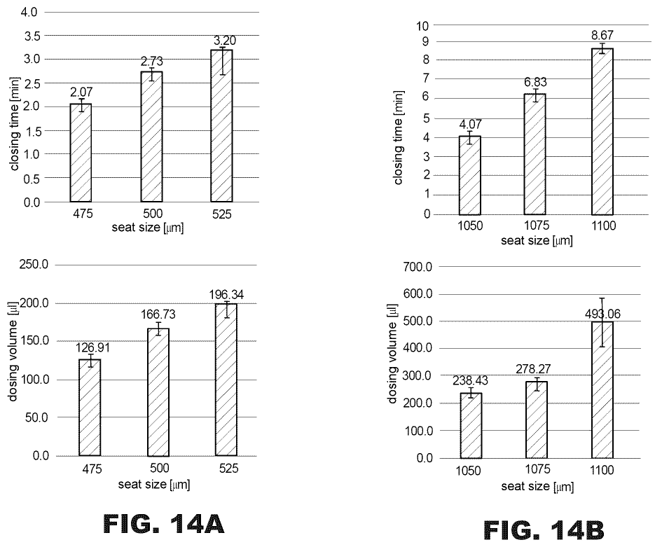

FIGS. 14A and B illustrate closing times for various closer elements 50. The closer elements were based on pDMAA (poly(N,N′-dimethylacrylamide)) with 10% BIS. To simulate an extraction protocol, a lysis/binding buffer flowed through a closer element for 2 minutes, and a washing buffer flowed through after 4 minutes until the closer element was completely closed. The pressure was 150 mbar at a flow rate of 100 μl/min, and the test was carried out 4 times for each size of closer element 4.

FIG. 14A illustrates the closing times and dosing volumes with lysis/binding buffer for various seat sizes. In each case, the closer element had a diameter of 600 μm and a height of 300 μm in the swollen production state.

FIG. 14B illustrates the closing times and dosing volumes with lysis/binding buffer for various seat sizes. In each case, the closer element had a diameter of 1200 μm and a height of 300 μm in the swollen production state.

In both test arrangements, a dependence of the closing time on the seat size is clearly apparent.

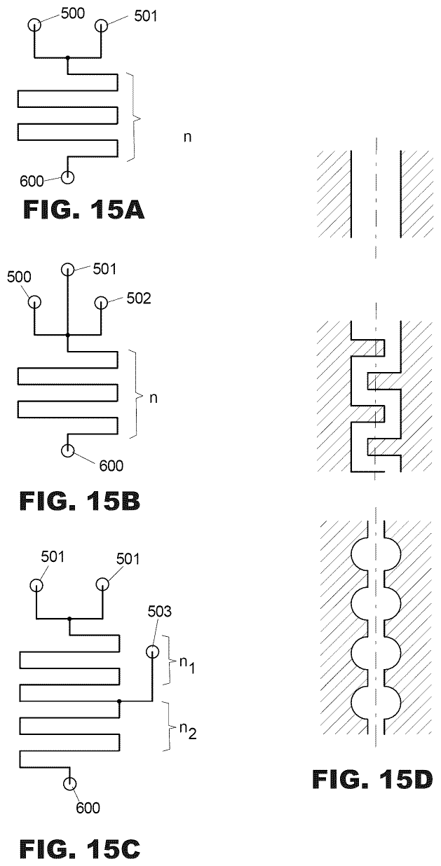

FIGS. 15A, B, C and D illustrate four different embodiments of the receiving region. FIGS. 11A-C each illustrate various embodiments of mixer structures. FIG. 15A illustrates a mixer structure for mixing two fluids comprising a meandering structure consisting of n turns. FIG. 15B illustrate a mixer structure for the simultaneous mixing of three fluids comprising a meandering structure consisting of n turns. FIG. 15C illustrates a mixer structure for mixing initially two fluids comprising a meandering structure consisting of n turns. A third fluid is then added to the fluids that have already been mixed, and is mixed with the two fluids that have already been mixed along a meandering structure consisting of n₂ turns. The fluids are added via the inlets 500-503, and the mixed fluid leaves the mixer via the outlet 600. FIG. 15D illustrates various channel structures. In each case, the channel centre line is indicated by a dashed line.

### Exemplary Embodiment 1

The apparatus according to the disclosure was used to produce a chip by means of the following protocol:
a. structure: 7 different layers
i. layer 1 and layer 3 consist of hydrophilic polyester (173 μm-thick) with an adhesive film on both sides (25 μm-thick in each case)
ii. layer 2 and layers 4 and 6 consist of PMMA (250 μm-thick)
iii. layer 5 consists of PMMA (4 mm-thick)
iv. layer 7 consists of PMMA (250 μm-thick with adhesive film (25 μm on one side)
v. the PTFE membrane is 100 μm-thick with 1.2 μm pores
vi. cellulose membrane (Whatman Grade 1) with a diameter of 1.2 mm (only a single ply is possible) or 3 mm (two-ply is also possible)
vii. layer 0 consists of hydrophilic polyester (is fixed on a glass slide-adhesion due to capillary forces (water droplets) or by means of an adhesive film (25 μm)
b. cleaning: apart from layer 0, all the layers are washed twice in an ultrasound bath for 15 minutes each with 0.1% Tween before assembly, followed by 2× double-distilled water (15 minutes), then dried with nitrogen and subsequently placed in a drying cupboard for 1 hour at 55° C. To remove any RNAs, washing is then carried out for 10 minutes with a cleaning solution, followed by three washing steps with DEPC water (with water treated with diethylpyrocarbonate). The layers are dried in an oven for 1 hour at 55° C.
c. manufacture of the chip: the hydrogels are first of all polymerized to obtain the closer elements on layer 0 under an inert gas atmosphere (argon). After washing of the closer elements with DEPC water and drying overnight at room temperature, optical alignment with layer 1 is performed. Both layers are manually laminated. The soluble polymers are then integrated to obtain the opener elements. After optical alignment and lamination, the steps are filled with PEG, and the cellulose membrane (silica membrane) is integrated. Layer 3 is then laminated by means of optical alignment. Layers 4 to 6 are aligned with one another by means of a tool with pins and are joined in a hot press at 10 kN for 25 minutes and at 65° C. The LAMP master mix is then integrated in the corresponding chambers. After the process of drying the LAMP master mix, the laminated layers 0-3 (part 1) and the compressed layers 4-6 (part 2) are optically aligned with one another and cold-compressed (2.5 kN). Layer 7 contains the PTFE membrane, which is then positioned relative to the rest of the chip by means of the pins and cold-pressed at 2.5 kN.

The apparatus according to the disclosure on the chip has a design in accordance with FIG. 12.

### Exemplary Embodiment 2

The chip produced in accordance with exemplary embodiment 1 was used as follows:
drying of the LAMP master mix: 1.4 mM dNTPs, 12 U hot start Bst2.0 (NEB), 5.7 U hot start RTx (NEB), 0.1% Tween, 1× primer mix (0.2 μM F3/B3, 1.6 μM FIB/BIP, 0.4 μM FL/BL) and 3 mM NaOH (in respect of final volume). Total volume=4.1 μl; reaction volume (after resuspension)=15 μl
LAMP chip with LAMP master mix was dried while standing for 2 hours at 35° C. to ensure that air exhaust channels were not clogged.
Simultaneous resuspension of the 6 reaction chambers with LAMP/elution buffer (spiked with Covid RNA BA5.0). Influenza A primer was dried as a control in the case of 3/6 reaction chambers
Closer elements close within one minute and prevent mixing of the master mixes
Yellow coloration only in the case of Covid-19 reaction chambers.

### Exemplary Embodiment 3

An apparatus according to the disclosure in accordance with FIG. 10 was operated as follows.

A fluid consisting of a lysis/binding buffer (200 μl) was introduced into receiving region 80. 100 μl of washing buffer containing 50% ethanol were introduced into receiving region 81, and water was introduced into receiving region 82 as an elution buffer. 3 minutes after the addition of the fluid, closer elements 50 and 54 closed. The closure of closure element 50 defined the volume of lysate. In addition, waste chamber 95 was closed by closer element 54. After 5 minutes, opener element 40 and opener element 44 opened. As a result, the washing buffer was able to flow out of channel section 110 into the first channel section 100 and, via the trap element 60, into waste chamber 220. Closer element 51 closed after 7 minutes, and opener elements 42 and 43 opened after 10 minutes. This enabled the elution buffer to flow out of channel section 111 into the first channel section 100 and, via the trap element 60 and opener element 43, into a region of the sample analyser 300. During this process, opener element 140 was activated. The first part of the eluate was collected in waste chamber 200 and immediately closed it upon contact with closer element 150. 2 minutes after the activation of opener element 140, it opened the flow path into the reaction chambers 210.

### Exemplary Embodiment 4

An apparatus according to the disclosure in accordance with FIG. 12 was operated as follows.

A lysate consisting of a VTM medium with Covid-19 RNA (24 μl) in lysis/binding buffer (24 μl RLTplus buffer+67 μl binding buffer+7 μl carrier-RNA) was introduced into receiving region 80. 100 μl of washing buffer (2.5 M NaCl, 10 mM tris-HCl, pH8) was introduced into receiving region 81, and elution buffer (10 mM ammonium sulphate, 50 mM KCl, 8 mM MgSO₄ in ddH₂O) was introduced into receiving region 82. There was a LAMP master mix in the reaction chambers 210. 3 minutes after the addition of the lysate, the inflow was stopped. Stopping the inflow of the lysate defined the volume of the lysate as 110 μl. After 5 minutes, opener element 40 opened. This enabled the washing buffer to flow out of channel section 110 into the first channel section 100 and, via the trap element 60, into the waste chamber 95. 7.7 minutes after the addition of the lysate, closer element 54 closed. In addition, the waste chamber 95 was closed by closer element 54.

Closer element 51 closed after 7.7 minutes, and opener elements 42 and 43 opened after 31 minutes. This enabled the elution buffer to flow out of channel section 111 into the first channel section 100 and, via the trap element 60 and opener element 43, into a region of the sample analyser 300. The eluate initially collected in the mixing chamber 240 and, after the filling of the mixing chamber, flowed into the reaction chambers 230 and, in this process, activated the closer elements 151, which closed within 1-2 minutes. The isolation efficiency for the E-gene and the S-gene of Covid-19 RNA was 98.1 and 88.9% respectively.

### LIST OF REFERENCE SIGNS

100, 110, 111, 112, 113 channel section
10, 11, 14 structure support
12, 13 cover
40, 42, 43, 44 opener element
40a channel-shaped part
40b, 40c connecting element
41 connection
50-54 closer element
60 trap element
61 grid
62 pillar structure
80-84 receiving region
90-94, 190, 191 permeable membrane
95 waste chamber
96 exhaust valve
140 opener element
150, 151, 152 closer element
192 exhaust valve
200 (waste) chamber
210 reaction chamber
220 waste chamber
230 reaction chamber
240 mixing chamber
300 sample analyser
500-503 inlet
600 outlet
a step width

1. Fluidic apparatus having a first channel section and n second channel sections, wherein n∈N and n≥1, at least one opener element and at least one closer element wherein
each of the n second channel sections is arranged so as to adjoin the first channel section by means of an opener element;
each opener element is configured to establish a fluidic connection from the respective second channel section to the first channel section;
the first and the n second channel sections each have a receiving region;
a closer element is arranged in the first channel section upstream of at least one of the opener elements in the flow direction of a fluid.

2. Fluidic apparatus according to claim 1, wherein the fluidic apparatus has n≥2 second channel sections and each of the n second channel sections is arranged spaced apart from one another in series on the first channel section.

3. Fluidic apparatus according to claim 1, wherein the fluidic apparatus furthermore has m third channel sections, wherein m∈N and m≥1, and
each of the m third channel sections is arranged so as to adjoin at least one of the n second channel sections by means of a respective opener element, and the respective opener element is configured to establish a fluidic connection from a third channel section to a second channel section.

4. Fluidic apparatus according to claim 3, wherein the fluidic apparatus furthermore has ƒ k^{th} channel sections, wherein ƒ∈N and ƒ≥1 and k∈N and k≥4
each of the ƒ k^{th} channel sections is arranged so as to adjoin at least one of the (k−1)^{th} channel sections by means of a respective opener element, and the respective opener element is configured to establish a fluidic connection from a k^{th} channel section to a (k−1)^{th} channel section.

5. Fluidic apparatus according to claim 1, wherein the first channel section furthermore has a trap element, which is arranged in the first channel section downstream, in the flow direction of a fluid, of the last opener element, which connects one of the n second channel sections to the first channel section.

6. Fluidic apparatus according to claim 5, wherein the trap element is a permeable element.

7. Fluidic apparatus according to claim 1, wherein the fluidic apparatus furthermore has one or more pressure and/or flow sources.

8. Fluidic apparatus according to claim 1, wherein the opener elements are soluble barriers.

9. Fluidic apparatus according to claim 1, wherein the closer elements are swelling material barriers.

10. Fluidic apparatus according to claim 5, wherein the fluidic apparatus furthermore has an apparatus for sample analysis, which is arranged downstream of the trap region of the first channel section in the flow direction of a fluid, and is fluidically connected to the first channel section.

11. Fluidic apparatus according to claim 10, wherein the apparatus for sample analysis is an apparatus for detecting/analysing analytes,
wherein the apparatus for sample analysis has j reaction chambers, wherein j∈N and j≥1;
wherein each reaction chamber has an inlet and an outlet; and
wherein a closer element is arranged upstream of the inlet of each reaction chamber in the flow direction of a fluid, and an exhaust valve and/or closer element is arranged at the outlet of each reaction chamber.

12. Fluidic apparatus according to claim 10, wherein the apparatus for sample analysis furthermore has a mixing chamber, which is configured to bring about equalization of concentration in a fluid.

13. Method for the sequential supply of defined fluid volumes by means of a fluidic apparatus having a first channel section and at least one second channel section, at least one opener element and at least one closer element,
wherein
the at least one second channel section is arranged so as to adjoin the first channel section by means of an opener element, and the opener element is configured to establish a fluidic connection from the at least one second channel section to the first channel section;
the first and the at least one second channel section each have a receiving region;
a closer element is arranged in the first channel section upstream of the opener element in the flow direction of a fluid;
the method comprising the following steps:
a) supply of a first fluid in the receiving region of the first channel section, thereby forming a flow direction of the first fluid in the first channel section;
b) supply of a second fluid in a receiving region of an at least one second channel section, thereby forming a flow direction of the second fluid in the at least one second channel section;
c) activation of the closer element by the first fluid in the first channel section and flow of the first fluid through the first channel section until the closer element is closed;
d) activation of the at least one opener element by the first fluid in the first channel section and/or activation of the at least one opener element by the second fluid in the at least one second channel section and opening of a fluidic connection between the first and the at least one second channel section;
e) inflow of the second fluid from the at least one second channel section into the first channel section and flow through the first channel section in the flow direction of the first fluid.

14. Method for the sequential supply of defined fluid volumes according to claim 13, wherein the fluidic apparatus furthermore has a trap element in the first channel section downstream of the opener element, and the fluids flow through it.

15. Method for the sequential supply of defined fluid volumes according to claim 13, wherein the fluidic apparatus furthermore has an apparatus for sample analysis, which is arranged on the first channel section by means of an opener element, wherein the opener element is configured to establish a fluidic connection from the first channel section to the apparatus for sample analysis;
wherein the apparatus for sample analysis is arranged downstream, in the flow direction of a fluid, of the opener element which connects the second to the first channel section, or is arranged downstream of the trap element in the first channel section;
and wherein a fluid flow in the first channel section activates the opener element, which fluidically connects the apparatus for sample analysis to the first channel section.
